(19) United States
(12) Patent Application Publication
(10) Pub. No.: US 2009/0017470 A1
(43) Pub. Date: Jan. 15, 2009

(54) Immunological analytical reagent for the determination of advance glycosylation end products (AGEs)

(76) Inventors: Yung-Hsiang Liu, Taipei city (TW); Wing-Yee Chan, Taipei city (TW)

Correspondence Address: BACON & THOMAS, PLLC, 625 SLATERS LANE, FOURTH FLOOR, ALEXANDRIA, VA, 22314-1176, US

(21) Appl. No.: 12/068,683

(22) Filed: Feb. 11, 2008

Related U.S. Application Data
(62) Division of application No. 10/867,693, filed on Jun. 16, 2004, now abandoned.

(51) Int. Cl. G01N 33/53

(52) U.S. Cl. 435/7.9; 436/501

(57) ABSTRACT

The invention provides a reagent for determination of Advance Glycosylation End Products (AGEs), comprising a suspension disposed with displaying carrier and the antigen or antibody immobilized on the surface of the said displaying carrier; and a test strip, comprising of: a base plate, and constitutive parts provided on said base plate, said constitutive parts consisting of a water absorption pad for the sample, a porous fiber membrane, a displaying carrier fiber block and at least one immobilized substance. After infusing the said reagent into the said test strip, the immunological reaction of the AGEs antigen or antibody can be determined based on the agglutination phenomenon or accompanied changes of absorbance or color and the presence or raised level of the AGEs in the diabetic patient can be known accordingly such that the practitioner can prevent the occurrence of the complicated condition, or block further the progression of the complications at the earlier stage.

This application is a divisional of application of U.S. Ser. No. 10/867,693 entitled “Immunological Analytical Method and Device for the Determination of Glycosylated Protein” filed on Apr. 16, 2004 and currently pending

### BACKGROUND OF THE INVENTION

1. Field of the Invention

The present invention relates to an immunological analytical reagent for the determination of the immunological reaction of antigen or antibody of Advance Glycosylation End Products (AGEs) by infusing a reagent into a test strip and observing agglutination phenomenon or accompanied changes of absorbance or color.

2. Description of the Related Art

When compared to its complications including kidney failure, hypertension, and ophthalmologic diseases, Diabetes itself is not so much life threatening. So far, the most basic test designated for diabetes is to measure the blood sugar levels before and after meal to find out if the patient suffers diabetes. When diabetes is confirmed, physician would usually rely upon blood test to monitor HbA1C levels to judge if the patient practices a satisfactory control of the blood sugar level. However, either the blood sugar or HbA1C assay can only test the said blood sugar level but fails to achieve earlier determination of the oncoming presence of diabetic complications. Even the microalbumin test available in the market may be used to determine kidney complication of a diabetic, the microalbumin test detects any kidney failure only after its incidence; meaning, the microalbumin test is rendered useless when comes to earlier preventive determination of the incidence of diabetic complications.

The pathogenic mechanism of diabetes has been thoroughly understood today. For example, it is known that, under the situation of hyperglycemia, a protein (e.g., albumin) might be glycosylated into Advanced Glycosylation End Products (AGEs), and this would cause abnormal destruction of cells, particular in a diabetic patient. Further, while the blood sugar level of a diabetic patient can currently be controlled effectively with drugs, the efficient manipulation of complicated condition through earlier prediction is still impossible.

The level of advance glycosylation end products (AGEs) in body fluid (e.g. blood) would be abnormally raised long before the formations of the complications of diabetics. Therefore, it has been devoted to develop a specific approach or agent to determine whether or not the AGEs of the diabetes is present and the attending practitioner could prevent the occurrence of the complicated condition in a diabetic patient at the earlier stage, or lower the concentration of the advance glycosylation end product (AGEs) in human blood to block the progression of the said complication. However, among such specific approach or reagent for determining the AGEs of the diabetes patient as an indication for earlier diagnosis, the most specific and sensitive one comprising of determining the AGEs antigen or anti-AGEs antibody, by immunological analytic techniques is not yet available at present.

### SUMMARY OF THE INVENTION

In view of the forgoing, the inventor of this application has been studied extensively, and finally developed an immunological analytical reagent for determining the AGEs antigen with the anti-AGEs antibody, and vice versa, which can determine whether any AGEs antigen or antibody is present or not in the sample based on the agglutination phenomenon due to the formation of the immunological complex between, for example, the anti-AGEs antibody and the AGEs antigen.

In another aspect, the invention provides an immunological analytical reagent for determining the AGEs antigen (anti-AGEs antibody) with the anti-AGEs antibody (AGEs antigen), which can determine whether any anti AGEs antigen or antibody is present or not in the sample based on the change of absorbance due to the formation of the immunological complex between, for example, the anti-AGEs antibody and the AGEs antigen.

The features, objectives and advantages of the invention will become apparent from perusal of the following description with reference to the appended figures in which:

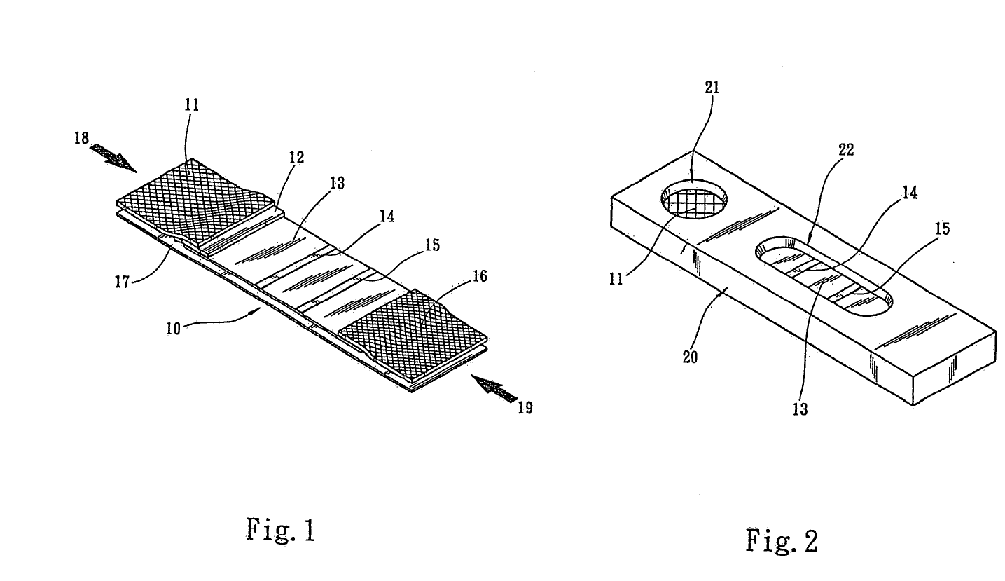

FIG. 1 is the three-dimensional structural view of the immunological chromatographic test strip according to the invention;

FIG. 2 is the three-dimensional outlined view of the box of a waterproof device, which box is used for accommodating the immunological chromatographic test strip shown in FIG. 1; and

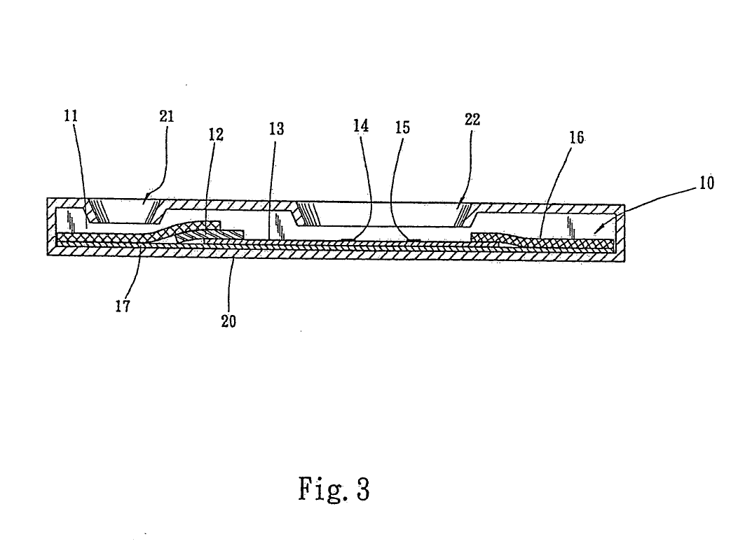

FIG. 3 is the overall exploded schematic view of the box of the waterproof device shown in FIG. 2, accommodating the test strip according to the invention.

### DESCRIPTION OF SYMBOLS

10 Test strip
11 Water-absorption pad
12 Displaying carrier fiber block
13 Porous fiber membrane
14 Reading zone
15 Reference zone
16 Absorption pad
17 Base plate
18 The front end of the test strip 10
19 The rear end of the test strip 10
20 Waterproof device box
21 Sample port
22 Port of the reaction zone

### DETAILED DESCRIPTION OF THE PREFERRED EMBODIMENT

As described above, the invention relates to the determination of the presence or not of an advance glycosylation end products (AGEs) in a sample to be tested by several immunological analytical techniques. Antibody used in the invention can be those raised immunologically in rabbit or goat with purified antigen (e.g., AGE antigen), or those obtained as hybridoma by immunizing mice (e.g., monoclonal antibody).

Immunological analytical techniques are known in the art. For example, Fujikawa H., and Igarashi, H. (Appl. Envir. Microbiol., 54/10, 2345-2348, 1998) disclosed flash suspension agglutination reagents formed from high density latex granules for detecting enterotoxin A˜E of Staphylococcus. Delanghe, J. R., Chapele, J. P., and Vander schueren, S. C., proposed a colorimetric method for detecting myoglobin (Clin. Chem., 36/9, 1675-1678, 1990). WO 88/08534 (1988) disclosed an immunological analytical device comprising an immunological chromatographic membrane as the medium for determining HCG and LH. U.S. Pat. No. 5,236,652 (1993) disclosed an immunological chromatographic technique for determining non-protein antigen.

None of the above-mentioned techniques taught about the immunological analytical technique for determining the AGEs antigen or AGEs antibody disclosed by the invention.

In one aspect, the invention provides an immunological analytic reagent for determining the AGEs antigen (antibody) with the anti-AGEs antibody (anti-AGEs antigen), which comprises of determining whether any AGEs antigen or antibody is present or not in the sample based on an immunological agglutination technique, which comprises:

A suspension disposed with displaying carrier, and an affinity substance immobilized on the surface of said displaying carrier and the said affinity substance is the AGEs antigen or antibody; which, after contacting said suspension reagent with a sample to be tested, can determine whether AGEs antigen or antibody is present or not based on the resultant agglutination phenomenon.

In one embodiment, one or more anti-AGEs antibody is immobilized on said displaying carrier in the suspension, and a blocking protein is used to fill fully voids in the carrier. When a test sample containing a given amount of AGEs antigen mixes and reacts with a displaying carrier suspension comprising one or more immobilized antibody on the reacting plate, immunological complexes as “displaying carrier-anti-AGEs antibody-AGEs antigen-anti-AGEs antibody”. Such reaction will result into a visible agglutination as a positive response within 3-5 minutes. On the other hand, if no AGE antigen present in the test sample, after mixing with the displaying carrier suspension comprising one or more immobilized anti-AGEs antibody, no agglutination will occur therebetween, and hence is considered as a negative response.

The displaying carrier used in the invention is a fine colored particulate with a particle size of 0.01-60 micron (a technical product already available from Bangs Labs Inc., USA). Such particulate may be any of latex micro-particles, dye micro-particles, liposomes, colloidal gold particles, carbon black micro-particles, and the like.

The term “antibody” as used herein refers to one or more of the above-mentioned anti-AGEs antibodies. Immobilization of an AGEs antigen having one structure or an AGEs antigen having various structures can be used to determine whether any anti AGEs antibody is present in the test sample or not; if no agglutination, it is a negative reaction; on the other hand, agglutination means positive reaction.

Alternatively, employment of the competitive method also achieves the same experimental results. For example, a competitor (e.g., a given amount of a known concentration of AGEs antibody or antigen is mixed with each sample pending test in an individual tube; each test tube is then added with displaying carrier suspension immobilized on its surface the AGEs antibody or antigen to react with the mixture for determination of the presence of AGEs antigen or antibody. In another aspect, the present invention provides a method to determine AGEs antigen or antibody using the analytical results of absorbance change by employing an immunology analytical reagent to test the AGEs antibody with the AGEs antigen, or vice versa, which comprises:

A displaying carrier suspension, immobilized on the surface of said displaying carrier with the above-mentioned anti-AGEs antibody or AGEs antigen; and

A device for measuring absorbance;

which, after contacting said reagent with a sample to be tested, can determine whether an AGEs antigen or antibody is present or not based on the resultant change or not of absorbance.

In one embodiment, one or more antibody is immobilized on said displaying carrier in the suspension, and a blocking protein is used to fill fully voids in the carrier. A negative serum standard, a weak positive serum standard, and a unknown test sample are prepared, respectively. To three calorimetric tubes A1, A2 and A3 are charged each of 250 micro-liter of displaying carrier suspension containing immobilized one or more antibodies. Then, adds 20 micro-liter of the negative serum standard into the colorimetric tube A1; adds 20 micro-liter of the weak positive serum standard into the calorimetric tube A2; and adds 20 micro-liter of the unknown sample into the calorimetric tube A3. Immediately after the additions are accomplished, the tubes are subjected to turbidity or absorbance measurement at 340 nm, and recorded the respective optical density (OD) value. At 240 seconds after addition of the sample, the respective OD values are recorded again. The difference by subtracting the first recorded OD values from the second recorded OD values, respectively, is the OD value of the immunological reaction. By comparing three OD differences thus obtained, when the difference of OD value from the unknown sample is greater than that from the weak positive serum standard, it can be considered as a positive response. On the other hand, when the difference of OD value from the unknown sample is less than that from the weak positive serum standard, it can be considered as a negative response. Furthermore, by preparing a series of standard solutions each having a know concentration, such as, for example, 0, 1, 2, 4, 8, and 16 units/ml, the concentration in the test sample can be calculated from the standard curve plotted from data obtained with such standard solutions.

The displaying carrier used herein is a fine particulate or enzyme (Banges Laboratories In, USA; Sigma, USA). Such particulates may be any of latex micro-particles, liposomes, polyethylene glycol micro-particulates, NAD micro-particles, carbon micro-particles, dye micro-particles, enzyme, NADH micro-particles, and the like with a particle size of 0.001-20 micron and a spectrophotometric range of 260 nm to 840 nm.

The term “antibody” as used herein refers to one or more of the above-mentioned anti-AGES antibodies. Immobilization of an AGEs antigen having one structure or an AGEs antigen having various structures can be used to determine whether any anti-AGEs antibody is present in the test sample or not. Alternatively, a competitive method can used to yield a same experimental result. For example, when conducted in the same manner as described above, additionally, a competitor (e.g., a given amount of AGEs antigen or antibody with known concentration) is mixed first with each sample pending test, positive standard solution, and negative standard solution in an individual test tube, and each test tube is then added with displaying carrier suspension immobilized on its surface the AGEs antibody or antigen to react with the mixture for determination of the presence of AGEs antigen or antibody by using a spectroscope to test the absorptivity variance of each individual tube to determine the presence of AGEs antigen or antibody.

Referring to FIG. 1, an immunological chromatographic test strip 10 comprises a porous fiber membrane 13 bonded on a base plate 17. A water absorption pad 11 for the sample is disposed at the most front end of the test strip 10, and is overlapped with the porous fiber membrane 13. A displaying carrier fiber block 12 is provided beneath the water absorption pad 11 for the sample and upon the porous fiber membrane 13 in a manner such that it is overlapped with them. The displaying carrier fiber block 12 has been impregnated with a blue displaying carrier and has an antibody or antigen immobilized thereon.

The color changes against the reference line due to the formation of the immunological complex between, for example, the anti-glycosylation end products antibody and the advance glycosylation antigen on a base plate. A reading zone 14 which may be in a dotted or linear distribution a spot or a line and which has antibody or antigen immobilized thereon is disposed on a section of the porous fiber membrane 13. The front end of the reading zone 14 faces the displaying carrier fiber block 12, while at an appropriate distance from its rear end, a reference zone 15 which may be in a dotted or linear distribution spot or a line is provided and which is also on a section of the porous fiber membrane 13 and which has antibody or antigen immobilized thereon. The reference zone 15 has its rear end facing an absorption pad 16. The material used to construct the above-mentioned porous fiber membrane 13 may be nylon fiber membrane, cellulose nitrate membrane, polyester fiber membrane, cellulose fiber membrane, synthetic fiber membrane and the like.

Referring to FIG. 2, a waterproof device box 20 for accommodating the immunological test strip 10 is consisted of a sample port 21 and a port of the reaction zone 22.

FIG. 3 is a three-dimensional view of the waterproof device box 20 accommodating a test strip 10. Wherein, the water absorption pad for the sample 11 is just below the sample port 21, and contact therewith. The area of the sample port 21 should be less than that of the water absorption pad for the sample 11. The reading zone 14 and the reference zone 15 are disposed within the port of the reaction zone 22, but do not contact therewith for visualizing them directly. The absorption pad 16 is located at the rear end of the device box 20. Since, as described above, the blue displaying carrier on the displaying carrier fiber membrane 12 has antibody or antigen immobilized thereon, upon contacting the blue displaying carrier on the displaying carrier fiber membrane 12 with the test sample, this blue displaying carrier will migrate freely on the test strip 10 in the direction toward the porous fiber membrane 13 and the absorption pad 16. The base plate 17 is associated on its lower side with the inner bottom side of the device box 20 and its upper side with the porous fiber membrane 13. The porous fiber membrane 13 is provided with a reading zone 14 and a reference zone 15, and has its voids filled fully with a blocking protein.

When a liquid test sample is added at the sample port 21, the test sample will react immediately with the antibody or antigen immobilized in the blue displaying carrier of the displaying carrier fiber block 12 and will migrate together with the blue displaying carrier toward the absorption pad 16. If an anti AGEs antigen is present in the test sample, it will react immediately with one or more antibodies of the anti-AGEs immobilized on the blue displaying carrier and occupy all of the epitope on the antibodies of the anti-AGEs. Since the binding sites of antibodies of the anti-AGEs immobilized on the blue displaying carrier will bind with the AGEs antigen and are occupied thereby, they shall not bind with the one or more AGEs antigen immobilized on the reading zone 14 such that all of the blue displaying carrier should pass the reading zone 14 and no visible blue line should appear on this zone. The blue displaying carrier will migrate further till bind with antibody or antigen (e.g., anti-rat immunoglobin G antibody) immobilized on the reference zone 15 to form a visible blue line. Therefore, a blue line on the reading zone 14 means a positive reaction, while a blue line will appear on the reference zone 15 in all cases disregarding a positive or negative reaction. On the other hand, if the test sample contains no target analyte, a portion of the one or more anti-AGEs antibody against the blue displaying carrier, will react with the AGEs antigen immobilized on the reading zone 14 and forms a visible blue line, while other portion of the blue displaying carrier will bind on the reference zone 15, i.e., a competitive reaction. Therefore, a blue line on the reading zone means a negative reaction. The absorption pad 16 is used for absorbing all of the liquid migrating to the end such that a capillary action can be sustained.

Alternatively, in the case as described above, a sandwich assay can be conducted just by replacing the AGEs antigen immobilized on the reading zone 14 with one or more anti-AGEs antibody. If an anti AGEs antigen is present in the test sample, it will react immediately with one or more antibodies of the anti-AGEs immobilized on the blue displaying carrier to form a “blue displaying carrier-anti-AGEs antibody-AGEs antigen” complex; the AGEs antigen of the said complex will migrate to the reading zone 14 to bind with one or more antibodies of the anti-AGEs immobilized on the blue displaying carrier and a blue line should appear on this zone, which is a positive reaction. The blue displaying carrier will migrate further till bind with antibody (e.g., anti-rat immunoglobin G antibody) immobilized on the reference zone 15 to form a visible blue reference line. If the test sample contains no AGEs antigen, the reading zone 14 will not appear a blue line, which is a negative reaction.

The porous fiber membrane used in the invention has a pore size in the range of 0.1-60 micrometer (Whatman Millipore). The displaying carrier used herein is a fine colored particulate, fluorescent substance or enzyme. Such particulates may have a particle size of 0.01-20 micron (Bangs Labs Inc, USA). The particle (Bangs Labs Inc USA; Sigma, USA), and may be any of latex micro-particles, dye micro-particles, liposomes, colloidal gold particles, carbon black micro-particles, or polymeric micro-particles in the present invention. When the displaying carrier is a fine colored micro-particle, the detecting result can be read directly and is referred as a direct displaying carrier. When the displaying carrier is an enzyme, the result can be read only after developing with a developing agent, and such displaying carrier is referred as indirect displaying carrier.

The base plate used in the invention is a waterproof plastic plate or waterproof paper. The materials used to construct the water absorption pad for the sample and the absorption pad used in the invention is not particularly limited, but it is better to have higher water absorbability. The displaying carrier fiber block is a water insoluble fibrous material.

The invention will be illustrated further more detailed with the following non-limiting examples.

### EXAMPLE 1

### Raising of the Antibody of the Advance Glycosylation End Products (AGEs)

The advance glycosylation ed products antigen is prepared as a polyclonal antibody by immunizing directly a rabbit or a goat with purified antigen, e.g., AGEs antigen, or by immunizing mice into hybridoma and further manipulating to yield as a monoclonal antibody.

### EXAMPLE 2

### The Agglutination Assay of the Advance Glycosylation End Products (AGEs) Antigen

A polystyrene bead or other colored micro-particles having a particle size of about 0.8 micrometer was used as the displaying carrier micro-particle and was diluted into a concentration of 3%. The AGEs antibody obtained in Example 1 above was diluted with a phosphate buffer into a concentration of 2 mg/ml. 10 ml each of the displaying carrier micro-particle suspension and the antibody solution were added into a glass tube and mixed well, which then stood for 8 hours. Thereafter, 1 g of bovine serum albumin (BSA) was added and mixed in the tube, which then stood for 8 hours. After centrifuging at 12000 rpm for 30 minutes, the supernatant was discarded, and repeated this operation once more. 2% BSA solution was added to a total amount of 20 ml. The mixture was sonicated into a homogeneous suspension as the desired displaying carrier suspension.

Three solutions of negative standard serum were prepared as having an AGEs antigen content of 0, 1 and 2 units/ml, respectively. A solution of weak positive standard serum was prepared as having an AGEs antigen content of 5 units/ml. A solution of strong positive standard serum was prepared as having an AGEs antigen content of 16 units/ml. Finally, an unknown test sample was provided.

100 microliter each of the serum solutions prepared above was placed into a test device having the test sample loaded, respectively. 50 microliter of the displaying carrier suspension was added into each test device, and read results after allowing them developing for 3-5 minutes.

### Results:

| | The displaying carrier |
|---|---|
| | agglutination suspension |
| Negative standard serum 0 unit/ml | — |
| Negative standard serum 1 unit/ml | — |
| Negative standard serum 2.5 unit/ml | — |
| Weak positive standard serum 5 unit/ml | + |
| Strong positive standard serum 16 unit/ml | + |
| Unknown test sample | + |

An agglutination reaction means a positive reaction for the AGEs antigen test, while no agglutination reaction means a negative reaction for the AGEs antigen test. With the minimum positive reaction limit set at 5 unit/ml, the positive reaction of the unknown test sample indicates the concentration of the AGEs antigen in this test sample is ≧5 unit/ml.

### EXAMPLE 3

### Immunological Turbidimetric Assay of the Advance Glycosylation End Products (AGEs)

0.2 g of the displaying carrier micro-particles was added into one liter of distilled water to prepare a suspension. The displaying carrier may be a white polystyrene bead having a particle size of about 0.3 micrometer. Alternatively, micro-particles having color at other wavelength can be employed also. To this suspension, 30 mg of the anti-AGEs antibody was added and mixed well, and then stood for 18 hours. Next, 4 g of BSA was added and mixed well, and again stood for 18 hours. The mixture was centrifuged at 12000 rpm for 30 minutes and the resulting supernatant was discarded. This process was repeated three times. 2% BSA solution was added to a total amount of one liter. The mixture was sonicated into a homogeneous suspension as the desired displaying carrier suspension.

Six standard serum solutions were prepared as having an AGEs antigen content of 0, 1, 2, 4, 8 and 16 units/ml, respectively. An unknown test sample was provided.

250 microliter of the displaying carrier suspension was added into a respective calorimetric tube. 20 micrometer each of the standard serum solution prepared above was added into each calorimetric tube, respectively. 20 microliter of the unknown test sample solution was added into another colorimetric tube.

The calorimeter was set to null with air at a wavelength of 340 nm. When 20 microliter each of the standard serum solution and unknown test sample were added, the OD value of individual colorimetric tube should read immediately and read again at 240 second thereafter. An OD difference of the reaction was calculated by subtracting the first OD value from the second OD value.

With the positive reaction limit set at 5 unit/ml, each reaction OD value obtained from each standard serum solution was plotted as a standard curve. The concentration of the AGEs antigen in the test sample can then be calculated from this standard curve and the resulting value is 9 unit/ml, a positive reaction.

### Results:

| | OD value | OD value | Reaction OD | |
|---|---|---|---|---|
| | at 0 sec | at 240 sec | value | Units |
| Standard serum A | 0.86 | 0.85 | 0.01 | 0 |
| Standard serum B | 0.92 | 0.87 | 0.05 | 1 |
| Standard serum C | 0.98 | 0.82 | 0.16 | 2 |
| Standard serum D | 1.02 | 0.83 | 0.19 | 4 |
| Standard serum E | 0.94 | 0.69 | 0.25 | 8 |
| Standard serum F | 0.99 | 0.69 | 0.3 | 16 |
| Unknown test sample G | 1.07 | 0.81 | 0.26 | 9 |

According to this example, negative serum solution having a concentration of 0 unit/ml and a positive serum solution having a concentration of 5 units/ml can be used to conduct a qualitative determination. If the reaction OD value of the unknown test sample is not less than the reaction OD value of the positive standard serum of 5 units/ml, a positive reaction is indicated. Otherwise, it is a negative reaction.

### EXAMPLE 4

### Assay of the Advance Glycosylation End Products (AGEs) Antigen with the Immunological Test Strip

A 3% suspension of blue displaying carrier micro-particles, which may be polystyrene beads, or other colored micro-particles having a particle size of about 0.3 micrometer, was prepared. A rabbit anti-AGEs antibody was diluted with phosphate buffer into a concentration of 2 mg/ml. 10 ml each of these was placed in a glass tube and mixed well, and then stood for 8 hours. 1 g of BAS was then added and mixed well, and then stood for 8 hours. The mixture was centrifuged at 12000 rpm for 30 minutes and the resulting supernatant was discarded. This process was repeated two more. 2% of BSA, and 10% of sucrose solution were added to a total amount of 20 ml. The mixture was sonicated to form a homogeneous suspension as the desired displaying carrier suspension.

A displaying carrier fiber block strip of 0.4×4.5 cm was impregnated in this displaying carrier suspension containing 10% sucrose, removed and dried in a desiccators at room temperature. After drying, it was dried in a freeze-dryer for 2 hours and then packed in a sealed bag containing desiccant and stored at 4° C.

A pre-determined amount of the displaying carrier suspension was loaded on the water absorption pad or on the front end of the porous fiber membrane; however, the latter is less favor for mass production.

An mouse anti-AGEs antibody (Mouse Antis-AGEs Ab) solution was spray-coated and immobilized on a band at 1.8 cm of a piece of long cellulose nitrate film of 15×4.5 cm, which spray-coated line is referred as the reading zone. Then, a solution containing anti-rabbit IgG was spray-coated and immobilized on the band at 3.4 cm, which spray-coated line is referred as the reference line. Thereafter, the cellulose nitrate film was impregnated in a phosphate buffer containing 5% BAS for at least 2 hours. The cellulose nitrate film was removed, rinsed with fresh water, and dried in a desiccator at room temperature. Thereafter, the film was stuck and completely covered a plastic base plate starting from the 2 cm position. The film thus treated was then dried in a freeze-dryer for 2 hours and then stored in a sealed bag containing desiccant at 4° C.

A water absorption pad for the sample of 15×3 cm was provided. The material of the water absorption pad for the sample or the water absorption pad is not particularly limited, but it is better for its higher water absorbability, and its dimension is variable.

The carrier fiber block was interposed between the front end of the porous fiber membrane and the base plate in a manner that the carrier fiber block was overlapped with the cellulose nitrate film. Then, the absorption pad for the sample was stuck on the carrier fiber block and the base plate in a manner that it was overlapped with the carrier fiber block. Finally, the absorbing pad was stick over the rear end of the finished porous fiber membrane and on the end of the base plate. At least some area of the absorbing pad was overlapped with the fiber membrane. Test strips with a width of 0.5 cm was cut therefrom as the finished product.

The finished test strip was installed in a waterproof device box in a manner that the sample port of this device box is at a position just above the water absorption pad for the sample, and contact therewith. The area of the sample port should be less than that of the water absorption pad for the sample. While the reading zone and the reference zone on the test strip should be in a visible position within the reaction zone of the device box.

The water absorption pad for the sample is disposed in front of the reading zone. The displaying carrier fiber block was overlapped one another with the cellulose nitrate and the water absorption pad for the sample. The reference line was behind the reading zone and the water absorption pad was behind the reference line. At 5 minutes after adding the test sample, the result can be read directly with the eye viewing through the reaction zone.

150 microliter of the negative serum sample was added in the sample port, the blue displaying carrier will migrate toward the reaction line (the reading zone and the reference line) through the capillary principle, and reached finally the water absorption pad at the last end. Since this negative sample did not contain the AGEs antigen, the anti-AGEs antibody immobilized on the blue displaying carrier would not react with the anti-AGEs antibody immobilized on the reading zone and hence no visible blue line would form thereon. The blue displaying carrier would bind with the anti-rabbit IgG antibody immobilized on the reference line and form a visible blue line, which meant a negative reaction. The absorption pad would absorb all of the liquid migrated thereto such the capillary action could be sustained.

When 150 microliter of a positive serum sample was into the sample port, the anti-AGEs antibody immobilized on the blue displaying carrier would bind with the AGEs antigen contained in the serum sample before the blue displaying carrier reached the reading zone. At the time the blue displaying carrier reached the reading zone, the anti-AGEs antibody immobilized on the reading zone would bind again with the AGEs antigen and formed a visible blue line. The remaining blue displaying carrier would pass the reading zone, and bind on the reference line, which exhibited a sandwich positive reaction.

Alternatively, by contacting the front section of the finished test strip product with the test sample for a period of time, an experimental result similar to that described above could be obtained thereby.

As described above, other than the sandwich approach, the above experiment can be conducted by a competitive method. According to Example 4, the experiment can be accomplished through a competitive method just changing the substance immobilized on the reading zone, for example, the AGEs protein antigen.

To determine the AGEs antibody, the substance immobilized on the blue displaying carrier is replaced with the AGEs antigen, and the immobilized substance in the reading zone is replaced with the AGEs antibody to perform the determination of anti-AGEs antibody using the competitive method. If the substance immobilized on the blue displaying carrier is related to the AGEs antigen, the substance immobilized in the ready zone is replaced with the anti-AGEs antibody or anti-human IgG antibody (referred as the second antibody) to perform the determination of anti-AGEs antibody using the sandwich method.

In conclusion, by testing with the specific reagent and the method according to the invention, the presence or not of the AGEs in the diabetes can be readily known so that the practitioner can, at the earliest stage, prevent the progression of the complicated conditions in the diabetic patient or blocking the further progression of the complicated conditions.

What is claimed is:

1. A reagent to determine Advanced Glycosylated End Products (AGEs) essentially comprising:
a suspension disposed with a displaying carrier; and
an affinity substance that is related to AGEs being immobilized on the carrier;
wherein, after the said reagent contacting the test sample, the presence of the AGEs antigen or anti-AGEs antibody in the test sample can be determined based on the occurrence of agglutination phenomenon.

2. A reagent as in claim 1, wherein said displaying carrier is a colored micro-particle.

3. A reagent as in claim 1, wherein the size of said displaying carrier is in the range of about 0.01 micrometer to 60 micrometer.

4. A reagent as in claim 1, wherein said affinity substance is an AGEs antigen to determine the anti-AGEs antibody in the test sample.

5. A reagent in claim 1, wherein said affinity substance is an anti-AGEs antibody to determine the AGEs antigen in the test sample.

6. A reagent to determine Advanced Glycosylated End Products (AGEs) essentially comprising:
a suspension disposed with a displaying carrier; and
an affinity substance that is related to AGEs being immobilized on the carrier;
wherein, after the said reagent contacting the test sample, the changes of absorbance being measured with an appropriate absorbance measuring device; the presence of the AGEs antigen or anti-AGEs antibody in the test sample can be determined based on the changes of absorbance.

7. A reagent as in claim 6, wherein said displaying carrier is a micro-particle or an enzyme.

8. A reagent as in claim 7, wherein said micro-particle is a micro-particle selected from the group consisting of latex micro-particle, liposome, dye micro-particle, polyethylene glycol micro-particle, NADH micro-particle, NAD micro-particle, polymeric micro-particle and carbon micro-particle.

9. A reagent as in claim 6, wherein said affinity substance is an AGEs antigen to determine the anti-AGEs antibody in the test sample.

10. A reagent as in claim 6, wherein said affinity substance is an anti-AGEs antibody to determine the AGEs antigen in the test sample.

11. A reagent to determine Advanced Glycosylated End Products (AGEs) essentially comprising;
a competitor;
a suspension disposed with a displaying carrier; and;
an affinity substance that is related to AGEs being immobilized on the carrier;
wherein, after the said test sample mixing the said competitor and then contacting the said displaying carrier, the changes of absorbance being measured with an appropriate absorbance measuring device; the presence of the AGEs antigen or anti-AGEs antibody in the test sample can be determined based on the changes of absorbance.

12. A reagent as in claim 11, wherein said affinity substance is an AGEs antigen and the competitor is AGEs antigen, to determine the AGEs antibody in the test sample.

13. A reagent as in claim 11, wherein said affinity substance is anti-AGEs antibody and the competitor is AGEs antibody, to determine the AGEs antigen in the test sample.

14. A reagent to determine Advanced Glycosylated End Products (AGEs) essentially comprising;
a competitor;
a suspension disposed with a displaying carrier, and;
an affinity substance that is related to AGEs being immobilized on the carrier;
wherein, after the said test sample mixing the said competitor and then contacting the said displaying carrier; the presence of the AGEs antigen or anti-AGEs antibody in the test sample can be determined based on the occurrence of agglutination phenomenon.

15. A reagent as in claim 14, wherein said affinity substance is an AGEs antigen and the competitor is AGEs antigen, to determine the anti-AGEs antibody in the test sample.

16. A reagent as in claim 14, wherein said affinity substance is anti-AGEs antibody and the competitor is anti-AGEs antibody, to determine the AGEs antigen in the test sample.
